(19) United States
(12) Patent Application Publication
(10) Pub. No.: US 2014/0336722 A1
(43) Pub. Date: Nov. 13, 2014

(54) METHOD AND NEUROPROSTHETIC DEVICE FOR MONITORING AND SUPPRESSION OF PATHOLOGICAL TREMORS THROUGH NEUROSTIMULATION OF THE AFFERENT PATHWAYS

(71) Applicants: CONSEJO SUPERIOR DE INVESTIGACIONES CIENTIFICAS (CSIC), Madrid (ES); GEORG-AUGUST-UNIVERSITAT GOTTINGEN, GOTTINGEN (DE)

(72) Inventors: Eduardo ROCON DE LIMA, ARGANDA DEL REY (ES); Jose Luis PONS ROVIRA, ARGANDA DEL REY (ES); Dario FARINA, GOTTINGEN (DE); Strahinja DOSEN, GOTTINGEN (DE)

(73) Assignees: CONSEJO SUPERIOR DE INVESTIGACIONES CIENTIFICAS (CSIC), Madrid (ES); GEORG-AUGUST-UNIVERSITAT GOTTINGEN, Gottingen (DE)

(21) Appl. No.: 14/271,669

(22) Filed: May 7, 2014

(30) Foreign Application Priority Data
May 8, 2013 (EP) 13382169.4

(51) Int. Cl. A61N 1/36; A61N 1/372

(52) U.S. Cl. CPC A61N 1/36139; A61N 1/36025; A61N 1/36067; A61N 1/37235
USPC 607/45

(57) ABSTRACT

Method and neuroprosthetic device for monitoring and suppression of pathological tremors in a user through the neurostimulation of the afferent pathways to the brain, which comprises wearable elements placed over the parts of the body affected by tremor, wherein said wearable elements (1) have sensors selected from bioelectric sensors (3) generating a bioelectrical signal characterization of tremor, biomechanical sensors (4) generating a biomechanical signal characterization of tremor and a combination thereof, a programmable electronic device (9) comprised of a control, acquisition and processing module of the characterization signals, and a signal generator for the afferent stimulation based on the bioelectrical, biomechanical or combination of both signal characterization and stimulation electrodes (8) which transmit the neuromodulation signals to the afferent pathways projecting into the central nervous system to modulate the activity of the neural structures responsible for tremor generation.

### OBJECT OF THE INVENTION

The present invention relates to a method and a neuroprosthetic device for monitoring and suppression of pathological tremor in a user via the stimulation of peripheral afferent pathways. The resulting afferent inflow projects into the central nervous system (spinal cord and brain) where it modulates the activity of the neural structures responsible for generating/maintaining pathological tremor. Therefore, with this method and device, functional compensation (i.e., attenuation) of the pathological tremor by the stimulation of the afferent pathways is achieved, in particular in the upper limbs.

The main fields of application of the present invention are the methods, material and electronic equipment within the orthoprosthetic and rehabilitation industries.

### BACKGROUND OF THE INVENTION

Pathological tremor can be defined as a strong involuntary oscillatory movement of a body part. Tremor is the most common movement disorder and its incidence and prevalence are increasing with the aging of the world population, affecting 15% of people aged between 50 and 89 years. The most common are tremors caused by the two neurodegenerative diseases: Parkinson's disease and Essential tremor. Although the disorder is not life-threatening, more than 65% of population suffering from upper limb tremor reports serious difficulties in performing the activities of daily living (ADL), greatly decreasing their independence and quality of life. The most common treatments are drug therapy or surgery. Patents and references described in this document are the current techniques for the treatment of tremor. However, 25% of patients do not benefit from any of these treatments, and this calls for the creation of alternative treatments to tremor.

The pathological tremors—simply referred to as tremors in the remainder of the document—arise due to various conditions. As a result, it is very difficult to differentiate tremors according to their etiology. Because their exact underlying mechanisms have not yet been clarified, none of them is fully understood. In the state-of-the-art, the tremors are treated with drug therapy or surgery, which may include thalamotomy or pallidotomy, or more commonly nowadays, thalamic stimulation (DBS). Unfortunately, both alternatives have significant drawbacks: drugs often have side effects, and show a decrease in effectiveness over the years of use (Olanow, C W et al. (2000) Preventing levodopa-induced dyskinesias, Ann Neurol 47, S167-S176; discussion 5176-168), while the DBS is associated with an increased risk of intracranial hemorrhage (˜4% of patients) (Kleiner-Fisman, G, et al. (2006). Subthalamic nucleus deep brain stimulation: Summary and meta-analysis of outcomes, Mov Disord 21, S290-S304) and psychiatric manifestations (Piasecki, S D et al. (2004) Psychiatric Complications of deep brain stimulation for Parkinson's disease, J Clin Psychiatry 65, 845-849), moreover, the percentage of eligible patients is very low (Perlmutter, J S et al. (2006) Deep brain stimulation, Annu Rev Neurosci 29, 229-257), for example, only 1.6 to 4.5% of the Parkinson's disease patients (Morgante, L, et al. (2007) How many parkinsonian patients are suitable candidates for deep brain stimulation of subthalamic nucleus? Results of a questionnaire, Parkinsonism Relat Disord 13, 528-531). Furthermore, the mechanisms accounting for the alleviation of tremor symptoms by drugs, thalamotomy or DBS are unknown, hampering the refinement of the existing treatment forms, and the development of novel ones.

Recently, the state-of-the-art has demonstrated the feasibility of managing upper limb tremors with biomechanical loading, applied either through robotic exoskeletons, or transcutaneous neurostimulation. This approach, on the contrary to pharmacotherapy or surgery, suppresses tremors by the modification of limb biomechanics, and not by targeting the site of tremor origin. In spite of attaining a systematic attenuation of moderate and severe tremors, there are a number of limitations in wearability, comfort, and selectivity (Rocon, E, et al. (2007) Design and validation of a rehabilitation robotic exoskeleton for tremor assessment and suppression, IEEE Trans Neural Syst Rehabil Eng 15, 367-378). These techniques are protected by various patents: ES200301767, ES200402966, U.S. Pat. No. 5,562,707, and U.S. Pat. No. 6,959,215 U.S. Pat. No. 7,643,882B2.

Recent experiments with subthreshold (afferent) transcutaneous stimulation carried out in the framework of TREMOR project (ICT-2007-224051) showed significant tremor attenuation in a number of patients (unpublished results). This finding is in agreement with a recent work on restoration of locomotion in rodent models of Parkinson Disease through spinal cord stimulation (Fuentes, R, Peterson, et al. (2009) Spinal cord stimulation restores locomotion in animal models of Parkinson's disease, Science 323, 1578-1582). Therefore, current evidences support the idea that the stimulation of the afferent pathways may alleviate the symptoms of tremors, probably by a disruption of low-frequency synchronization in the corticobasal ganglia circuits (Fuentes, R, et al. (2010) Restoration of locomotive function in Parkinson's disease by spinal cord stimulation: mechanistic approach, Eur J Neurosci 32, 1100-1108).

Within the state-of-the-art, there are several patents that describe different techniques of stimulation but none protects the concept of this invention. The patent US20060217781A1 (Systems and methods for treating disorders of the central nervous system by the modulation of brain networks) describes a series of methods and systems for the treatment of brain disorders through the neuromodulation of brain structures. The patent describes a number of methods for implementing neuromodulation (electrical, optical, magnetic or chemical stimulation) of the structures of the brain associated with the disease. Patent US20060212089 (Device for the desynchronization of neuronal brain activity) focuses on the description of a device for the desynchronization of brain areas. Importantly, all of the proposed techniques are focused on the direct stimulation of the brain and not on the stimulation of peripheral afferent pathways (sensory nerves), as described in this patent. The patent application US2011184489 entitled “Method of Treating Parkinson's Disease and Other Movement Disorders” describes a method for the treatment of disorders associated with Parkinson's disease based on electrical stimulation of the spinal cord. Patent WO2010141155A1 entitled “Selective neuromodulation using energy-efficient waveforms” describes methods for selective neuromodulation in mammals.

The patent application WO2005122894 “Method and electronic computing device for suppressing and evaluating tremors and spastic movements in relation to input and control peripherals” describes a method and device for discerning the tremor movement generated by neurological disorders.

### DISCLOSURE OF THE INVENTION

The present invention discloses a method and a neuroprosthetic device for monitoring and suppression of pathological tremor in a user via neurostimulation of the peripheral afferent pathways (sensory nerves). This invention is employed as an alternative treatment to the reduction of tremor in patients suffering from the pathologies that generate tremor, in particular Essential tremor and Parkinson Disease. Its industrial application takes the form of a neuroprosthesis (implanted or transcutaneous) to reduce the tremor through the neuromodulation of the afferent pathways. The final product therefore is a neuroprosthesis, integrating all the electronics for the control, acquisition and processing of tremor variables acquired by bioelectrical sensors (e.g., EMG) and/or biomechanical movement sensors (e.g., inertial measurement units). This electronics is preferably integrated into a textile substrate to be worn on the human upper limb, with a set of electrodes (implantable or transcutaneous) for neurostimulation. The neuroprosthetic device, in addition to suppressing tremor, may be a tremor-monitoring tool during the patient's daily life. Furthermore, the proposed method allows adapting automatically the neuromodulation strategy to the different conditions of tremor movement in each patient.

A first object of the present invention is a neuroprosthetic device for monitoring and suppression of pathological tremor in a user via stimulation of the afferent pathways. This device is preferably portable. This concept could either replace or complement the traditional pharmacotherapeutic treatment of tremor. The stimulation is closed-loop controlled by a series of sensors that quantify the tremor of the person. The sensor data are processed and used to define adequate levels and timing of the stimulation.

As a result, the neuroprosthetic device comprises at least one part worn by the user and located on the part of the body affected by tremor. The part worn by the user may be preferably a textile garment and placed on the user's arm or forearm, but it could also be placed on the other areas of the body. Each of the wearable elements comprises:
At least one sensor selected from at least one bioelectrical sensor that generates a bioelectrical characterization of tremor, at least one biomechanical sensor that generates a biomechanical characterization of tremor and a combination thereof;
A programmable electronic device comprising at least one module for the control, acquisition and processing of the electrical, biomechanical or both signal characterizations, and an electric signal generator for generating a signal of the afferent stimulation from the electrical, biomechanical or both characterization signals and,
At least one stimulation electrode integrated into the wearable textile that transmits the signal for the stimulation to the afferent pathways.

In a particular implementation of the invention, each of the wearable elements comprises:
At least one bioelectrical sensor that generates a bioelectrical characterization of tremor;
At least one biomechanical sensor that generates a biomechanical characterization of tremor;
A programmable electronic device comprising at least one module for the control, acquisition and processing of the electrical and biomechanical signal characterizations, and an electric signal generator for generating a signal of the afferent stimulation from the electrical and/or biomechanical characterization signals and,
At least one stimulation electrode integrated into the wearable textile that transmits the signal for the stimulation to the afferent pathways.

The combination of at least one bioelectrical sensor and at least one biomechanical sensor provide additional input for online tremor detection and analysis. This can be used as a redundant input, making the device more robust. Additionally, the biomechanical sensors can provide information when the bioelectrical sensors, such as EMG, cannot. For example, while the stimulation is being delivered (EMG corrupted by artifacts).

In a particular implementation of the invention the neuroprosthetic device comprises at least one electroencephalography sensor that measures brain activity and generates a signal that characterizes the intention of the user to move the limb affected by tremor.

In another particular implementation of the invention, the device comprises an external device connectable to a programmable electronic device with an interface for accessing the functionalities of at least one wearable element. In a more particular implementation of the invention, the target programmable electronic device can be a computer, a tablet, a smartphone or a similar device. It is also envisaged that in another particular implementation of the invention, the external device comprises means for displaying measurements taken by the neuroprosthetic device based on the acquired signals, a processor for analysis and generation of diagnostics and reports of tremor and a memory to store the measurements performed by the neuroprosthetic device.

In another particular implementation of the invention, the at least one stimulation electrode is an electrode selected between an implanted electrode (e.g., intramuscular wire electrode) and a transcutaneous electrode.

In another particular implementation of the invention, the neuroprosthetic device comprises at least one bioelectric sensor selected from a classic multichannel bipolar EMG, high-density electromyography sensors (hdsEMG), electroencephalography sensors (EEG), or a combination of those, measuring electrical activities of the muscles and brain and characterizing the pathological tremor and/or voluntary movement intentions of the user.

In another particular implementation of the invention, the neuroprosthetic device comprises at least one biomechanical sensor is an inertial sensor selected from gyroscopes, accelerometers, magnetometers or a combination of those.

In another particular implementation of the invention, at least one electromyography sensor is a multichannel thin film electrode.

In another particular implementation of the invention, at least one stimulation electrode is a thin film multi-channel electrode.

In another particular implementation of the invention, the connection between the programmable electronic device and the external device is selected from a wireless connection or a wired connection.

In another particular implementation of the invention, the neuroprosthetic device as described in this patent application, further comprises a programmable electronic device selected from a computer, a smartphone and a tablet device, the connection between this programmable electronic device and the external device is selected from a wireless connection or a wired connection, and the external device comprises means for displaying measurements taken by the neuroprosthetic diagnostic device based on the available sensors, a processor for analysis of tremor and generation of diagnostics and reporting, and a memory storage for the measurements made by the neuroprosthetic device.

Therefore, the present invention discloses an ambulatory neuroprosthesis, preferably wearable, to remotely monitor and treat tremor during the user's daily life, where the neuroprosthesis integrates a neural stimulation and a high resolution measurement systems; these two components are integrated into the neuroprosthesis object of this invention. As a result, with the measurement and stimulation systems integrated into the neuroprosthetic device, the system allows to merge neural and biomechanical information to achieve a correct parameterization of tremor in everyday life, and use this information to control the closed-loop stimulation for tremor suppression. Additionally, this data will be interpreted in order to assess the tremor, and provide both to the neurologist and the individual patient the metrics reflecting the current status and evolution of the tremor. This will facilitate remote monitoring of treatment. In addition, the patient will get more involved in the therapy. It will also allow an objective comparison regarding a long-term drug therapy, which is the current gold standard for the tremor treatment.

The neuroprosthesis concept provides a novel way to manage the involuntary movements in the part of the body affected by tremor. The system will be easy to use and self-contained; this means that the acquisition, control electronics and power supply will be incorporated into the neuroprosthetic device, providing functional treatment of the most common forms of tremor in upper limb, for instance, those caused by Parkinson and Essential tremor. It will facilitate the independence of the patients, and maximize their time out of the care centers, saving costs associated with medical care.

A second object of the present invention is a method of monitoring and suppression of pathological tremor in a user via the stimulation of peripheral afferent pathways, utilizing the neuroprosthetic device described above. This method comprises the following steps:
i) Tremor characterization by a plurality of sensors selected from bioelectrical sensors generating a bioelectrical characterization signal, biomechanical sensors generating a biomechanical characterization signal and a combination thereof, wherein these tremor characterization signals comprise at least frequency, phase or amplitude of tremor;
ii) To provide the programmable electronic device with these tremor characterization signals;
iii) To distinguish the tremor from the voluntary movement of the user through a conventional analysis of the bioelectrical characterization signal, the biomechanical characterization signal or the combination of bioelectrical and biomechanical characterization signal;
iv) To calculate the neurostimulation control signal from the bioelectrical, biomechanical or both characterization signals, which is implemented in the module for control, acquisition and processing of the programmable electronic device;
v) To generate the neurostimulation signal through the electronic signal generator (i.e., electrical stimulator) of the programmable electronic device and,
vi) To send the stimulation signals to the plurality of neurostimulation electrodes that will stimulate the afferents pathways.

In a particular implementation of the invention, the method of monitoring and suppression of pathological tremor in a user via the stimulation of peripheral afferent pathways, utilizing the neuroprosthetic device described above. This method comprises the following steps:
i) Tremor characterization by a plurality of bioelectrical and biomechanical sensors generating a bioelectrical characterization signal and a biomechanical characterization signal wherein these tremor characterization signals comprise at least frequency, phase or amplitude of tremor;
ii) To provide the programmable electronic device with these tremor characterization signals;
iii) To distinguish the tremor from the voluntary movement of the user through a conventional analysis of the bioelectrical characterization signal and the biomechanical characterization signal;
iv) To calculate the neurostimulation control signal from the bioelectrical and biomechanical characterization signals, which is implemented in the module for control, acquisition and processing of the programmable electronic device;
v) To generate the neurostimulation signal through the electronic signal generator (i.e., electrical stimulator) of the programmable electronic device and,
vi) To send the stimulation signals to the plurality of neurostimulation electrodes that will stimulate the afferents pathways.

In another particular implementation of the invention, when the device has at least one electroencephalography EEG sensor which measures the brain activity of the user, the method further comprises the following steps:
To add to the signals measured in step i) the user's brain activity as captured by at least one EEG sensor and generate an electroencephalography signal characterising the intended (voluntary) user movement of the body part affected by the tremor;
To provide electroencephalography signals, in phase ii) to the control, acquisition and processing module of the programmable electronic device;
Additionally, to use electroencephalography signal in step iv) to calculate the neurostimulation signal to be sent to the neurostimulation electrodes for the activation of the afferents pathways.

In another particular implementation of the invention, stimulation of the afferent pathways disrupts the low-frequency synchronization in the corticobasal ganglia circuits of the user.

In another particular implementation, the afferent inflow generated by the stimulation of the peripheral afferent pathways projects into the central nervous system (spinal cord and brain) where it modulates the activity of the neural structures responsible for generating/maintaining pathological tremor. The latter will result with tremor attenuation.

In another particular implementation of the invention, the analysis employed by the programmable electronic device for discriminating the tremor from the voluntary movement is based on algorithms in the time and frequency domain. More preferably, the algorithm used is the iterative Hilbert transform. An implementation of this type of signal analysis to differentiate tremulous from the voluntary movement of the patient is described in the patent application WO2005122894 (Method and electronic computing device for suppressing and evaluating tremors and spastic movements in relation to input and control peripherals). However, one could employ any technique, which allows clear differentiation of the signal generated by a tremor from the signal generated by a voluntary movement of a user that suffers from a neurological pathological condition.

In another particular implementation of the invention, the programmable electronic device sets:
The parameters of neuromodulation strategies to be executed, and
The operating mode of the neuroprosthetic device.

In another particular implementation of the invention the calculation of neurostimulation signal in the programmable electronic device is based on an algorithm in the frequency or time domain. More preferably, the algorithm in the frequency or time domain is selected from an IEEE-1057 standard algorithm, a Kalman Filter and Benedict-Bordner filter.

Therefore, in summary, the proposed pathological tremor monitoring and suppression methods consist of two main stages:

The first stage is a strategy for identifying the characteristics of the tremor from information gathered from a variety of biomechanical (inertial sensors) and/or physiological (EMG or EEG signal) sensors. The features obtained by this strategy are at least the frequency, phase, amplitude of the tremor signal or a combination thereof. The method also allows differentiating the pathological tremor signal from the voluntary signal by means of any digital algorithm or any analog or digital electronic circuit designed for this purpose. Furthermore, information can be obtained on the cerebral activity, for example during preparation and initiation of a voluntary movement of the patient through the EEG signal. Additionally, the characteristics obtained from tremor may be used to monitor, track and log tremor during the patient's daily life.

The second stage consists of a control loop for the neuromodulation of afferent pathways, which uses the tremor information obtained in the previous phase, to define the intensity and timing of electrical stimulation of the afferents pathways in order to relieve the symptoms of tremor, preferably, by interrupting the synchronization of tremor-related oscillations at spinal level or brain, thereby preventing the development of tremor. In this context, two possible control strategies are provided that can be implemented by the method and device object of this patent, both based on the neurophysiological tremor origin:
A first strategy is a desynchronization of the tremorogenic central network by means of afferent neurostimulation. The rationale for this is that Essential tremor and Parkinson's disease are caused primarily by the brain structures that exhibit an abnormal oscillatory activity. Although a detailed description of such networks is still lacking, it is accepted that the desynchronization of this abnormal activity is the mechanism that mediates tremor management with deep brain stimulation, and also likely, through pharmacotherapy. In addition, the delivery of afferent stimuli to the supraspinal centers from the periphery has recently been shown to improve the symptoms of Parkinson's disease. The neurostimulator defined in this patent will directly stimulate the nerves innervating the tremulous muscles. This stimulation will be reaching the central tremorogenic network without interfering with other neural processes. To this end, the neurostimulator will stimulate in a range of frequencies aimed at causing the desynchronization of the tremor network.
The second strategy is the decorrelation of the common input to motor neurons that innervate the affected muscles. This approach relies on the hypothesis that the desynchronization of the common tremor input to the motor neuron pool may reduce tremor amplitude. The rationale for this is that a secondary input, commonly projected to a pool of motor neurons, hampers the transmission of a primary input—in this case the tremor. Indeed, this technique is based on today treatments known to alleviate the symptoms of Parkinson's disease by vibrating the whole body. Interestingly, the experiments showing that limb cooling reduces the severity of tremor arising from Parkinson's disease, essential tremor, and multiple sclerosis, also provide support of this approach.

Furthermore, by using an Internet connection, the neuroprosthetic device can send objective information for the physician to monitor in real time (online) the effects of treatment for tremor suppression.

The main advantages of the method and device objects of this invention compared to other techniques and devices within the state of the art could be summarized as:
It is portable and ambulatory.
It is less invasive than DBS and the percentage of eligible patients is higher.
It is the first technique based on the afferent stimulation for minimizing the tremor. This holds several advantages with respect to the classical application of electrical stimulation where the muscles are activated to produce tremor canceling forces. Since in our approach the stimulation is below the motor threshold (afferent stimulation), the current intensities are lower, there is no significant muscle contraction and the stimulation is thereby more comfortable and does not produce muscle fatigue.
The concept proposed in this patent will replace or complement the pharmacological treatment of tremor.
The device will allow monitoring tremor of the patient under any treatment, which could be based on the neuroprosthetic device described in this document or conventional treatment (pharmacotherapy or surgery).
It does not limit the range of movements and freedom of the patient.

### BRIEF DESCRIPTION OF THE FIGURES

FIG. 1.—Illustrates schematically the architecture of a particular implementation of the neuroprosthetic device defined in the present invention.

FIG. 2.—Illustrates an example implementation of the device object of the invention placed on a limb of a user.

FIG. 3.—Illustrates an example implementation of the method for monitoring and suppression of pathological tremors defined in the present invention.

FIG. 4.—Illustrates the components of the device presented in Example 1: (1) Bioelectrical sensor (i.e., EMG amplifier and Ag/AgCl electrodes), (2) Programmable electronic device with processing module (i.e., standard laptop with custom-made control software) and acquisition module (i.e., USB data acquisition card), and (3) electronic signal generator (i.e., programmable stimulator). EMG from the wrist and finger flexors and extensors was recorded, processed to estimate tremorogenic activation, and the stimulation was delivered to the same muscles out of phase with the tremorogenic bursts above and below the motor threshold.

FIG. 5.—Illustrates the device operation of the device presented in Example 1. EMG from the wrist and finger flexors and extensors was recorded and used as input to an Iterative Hilbert Transform based algorithm estimating the tremorogenic EMG bursts. Based on this, the bursts that were expected to appear within the next stimulation window were predicted and the stimulation was delivered out of phase with the predicted EMG bursts. The rectangles in the figure are the steps of the algorithm, the full arrows represent the data flow, and the dashed arrows are the input parameters. The rectangle with a thick edge denotes the initial step. Notation: MIBI—mean inter-burst interval.

FIG. 6A.—An example of the flexor processing presented in FIG. 5. This output is shown online to the device operator.

FIG. 6B.—An example of the extensor processing presented in FIG. 5. This output is shown online to the device operator.

FIG. 7A.—Wrist flexion/extension joint angle recorded from subject ET1 while the device was active delivering the stimulation below the motor threshold [device on-afferent stimulation]. The signals were created by concatenating data segments from several trials in the aforementioned conditions.

FIG. 7B.—Wrist flexion/extension joint angle recorded from subject ET1 while the device was active delivering the stimulation above the motor threshold [device on-motor stimulation]. The signals were created by concatenating data segments from several trials in the aforementioned conditions.

FIG. 7C.—Wrist flexion/extension joint angle recorded from subject ET1 while the device was off [device off]. The signals were created by concatenating data segments from several trials in the aforementioned conditions.

FIG. 8.—Wrist flexion/extension joint angle during stimulation below the motor threshold. Stimulation and recording windows are separated by the vertical dashed lines. As it is seen, tremor was attenuated during the stimulation with respect to the recording. The degree and consistency of suppression was however variable.

FIG. 9A.—Frequency analysis of tremor suppression: power spectral density of the wrist angle signals recorded during the delivery of above motor threshold stimulation (motor stimulation). The plot (9A) compares the system on (stimulation and recording together) versus system off conditions.

FIG. 9B.—Frequency analysis of tremor suppression: power spectral density of the wrist angle signals recorded during the delivery of above motor threshold stimulation (motor stimulation). The plot (9B) compares the tremor power for the signals recorded during the stimulation and recording time intervals separately with respect to the periods when the system was off. There was an overall suppression during above threshold stimulation and during both recording and the actual stimulation delivery.

FIG. 9C.—Frequency analysis of tremor suppression: power spectral density of the wrist angle signals recorded during the delivery of below motor threshold stimulation (afferent stimulation). The plot (9C) compares the system on (stimulation and recording together) versus system off conditions.

FIG. 9D.—Frequency analysis of tremor suppression: power spectral density of the wrist angle signals recorded during the delivery of below motor threshold stimulation (afferent stimulation). The plot (9D) compares the tremor power for the signals recorded during the stimulation and recording time intervals separately with respect to the periods when the system was off.

There was an overall suppression during below threshold stimulation and during both recording and the actual stimulation delivery.

### EXAMPLES

This section provides a description of several implementations of the invention, with an illustrative and non-limiting approach, making reference to the numbering adopted in the figures.

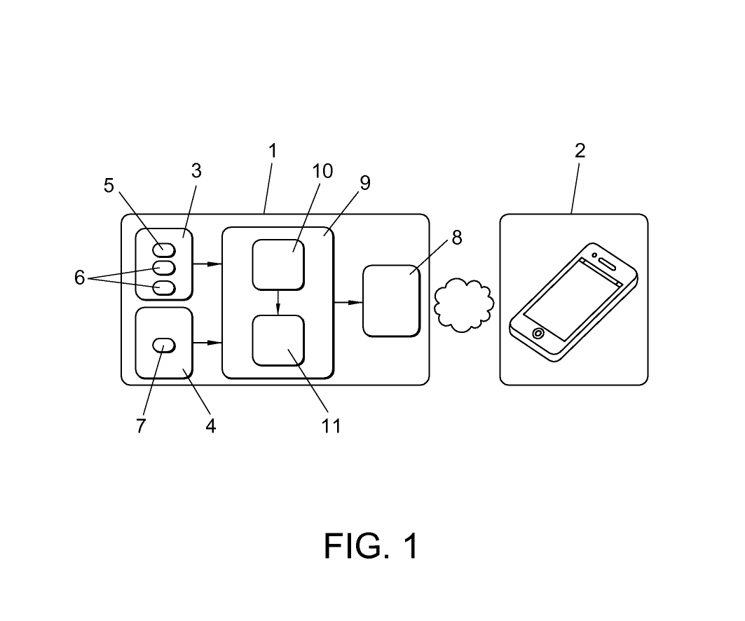

FIG. 1 shows an implementation of the neuroprosthetic device composed of the wearable garment (1), in this case textile, and the connectable external device (2) from where the operation of the neuroprosthesis and the tremor characteristics are monitored.

This neuroprosthesis, which will be useful for monitoring, diagnosis and pathological tremor suppression in any of its variants, is being composed of the following components:
Wearable garment (1) that enables the integration of inertial sensors, and electronics for neurostimulation and electrophysiological signal acquisition and processing, together with a module for data storage and communication. This item (1) provide long-term records of neural and biomechanical data of the body part to which it is placed, and treats the tremor during the patient's daily life by delivering the afferent stimulation. The collected information is analyzed, and derived metrics are extracted to provide both the patient and clinician with feedback of the therapy progress. The clinician will have remote access to this data, and will be able to adjust the therapy, and the user will be involved in the treatment.
Bioelectric sensors (3):
   a. Electrodes to measure surface (EMG) or intramuscular (iEMG) electromyography (6). These may be bipolar or high-density electrodes (6).
   b. Electrodes to measure electroencephalography (EEG) (5).
The information from the bioelectric sensors may be useful for the management of control strategies for neurostimulation.
Motion sensors (4) such as inertial sensors (IMUs) (7)
Electrodes (transcutaneous or implantable) for neurostimulation (8).
A programmable electronic device (9) which may be a microcontroller, microprocessor, DSP, . . . computer, which is responsible for:
   a. The identification and tracking of the tremor motion using appropriate state-of-the-art algorithms.
   b. The implementation of control algorithms for neurostimulation.
   c. The acquisition of the sensors signals (3, 4) of the wearable element (1).
   d. The generation of signals for neurostimulation.
   e. Communication with an external device (2). The programmable electronic device (9) is able to communicate both wirelessly (Bluetooth, Zigbee, WiFi or IrDA) or through cable connection (TCP/IP, serial cable) with the external device (2).
An external device (2) (PC, iDevice (iPhone, iPad), Smartphone or similar) responsible for:
   The implementation of an interface to access all the features of the neuroprosthesis. This interface allows:
      i. The adjustment of the parameters of the control strategies for neurostimulation.
      ii. The definition of the operation mode of the neuroprosthesis.
      iii. The presentation of the variables measured by the neuroprosthesis.
      iv. The analysis of the tremor motion.
      v. The presentation of the machine-based diagnostic.
      vi. The storage of the data acquired by the neuroprosthesis.
      vii. The preparation of reports.
   The communication (wirelessly or via a cable connection) with the neuroprosthesis.

For the case in which the electrodes for neurostimulation are implantable (8), all the electronics would be integrated into the textile and only the electrode (intramuscular thin film or wire) would be implanted in the muscle. For the case in which the electrodes for neurostimulation are transcutaneous (8), all the electronics and the electrodes would be integrated into the textile.

The programmable electronic device (9) comprises the entire electronic for control, acquisition and processing of the signals acquired by the sensors. The electronic device (9) is also running different calibration strategies for the control and suppression of tremor and it is capable of generating the appropriate signal for the afferent neurostimulation based on the information gathered by the sensors (3,4) and it is also able to communicate with the external device (2). This external device (2), which is connected to the programmable electronic device (9), is able to provide an interface to access all the features of the neuroprosthesis (1) and present data in a convenient way to the users, who could be a patient or a clinician.

The sensors, biomechanical and bioelectrical (3, 4), would be used to extract information about the patient's movement and, using the algorithms programmed into the neuroprosthesis, extract features of patient's tremor. These tremor features allow customization of stimulation applied to each patient.

The use of this neuroprosthetic device is twofold. On one hand, it can be used as a tool for tremor evaluation and objectification in patients under normal conditions or under any tremor suppression treatment (pharmacological or surgical) and, on the other hand, as a tool for the treatment of these patients with tremors.

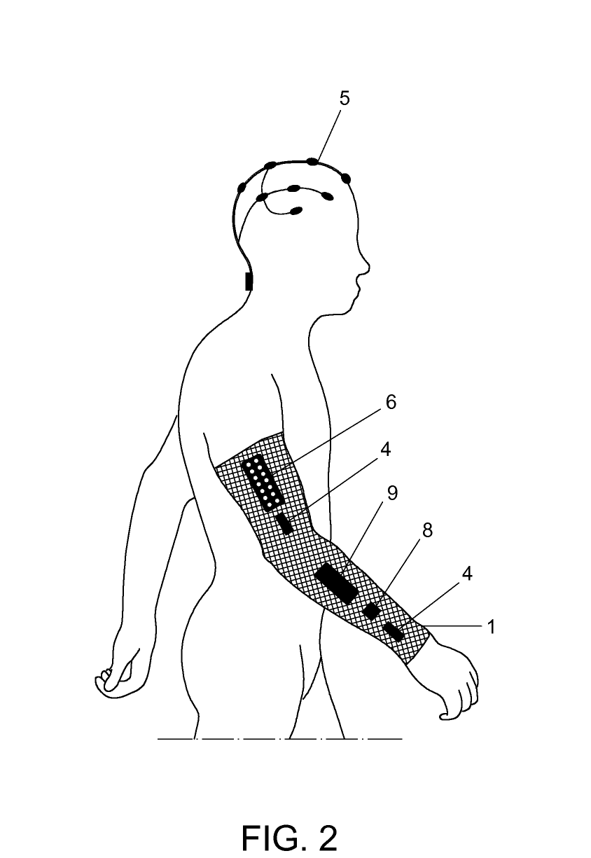

FIG. 2 shows another implementation of the neuroprosthetic device object of the present invention in which the wearable garment is placed on the arm and forearm of a user. Therefore the wearable element (1), which in this case will be textile, integrates bioelectrical sensors (3), movement sensors (4), a programmable electronic device (9) and neurostimulation electrodes (8).

This example is based on the adoption of two gyroscopes as motion sensors (4). These sensors are integrated into a flexible substrate, and are therefore very convenient for the integration within the garment.

Both gyros are placed on the textile structure on both sides of the wrist joint and provide information about the absolute angular speed of the segments (hand and forearm) in the plane of motion of the joint. The sensors measure the frequency range between 0 and 50 Hz, thereby covering the entire frequency range of possible tremors.

Bioelectric sensors (3) used in the example for the measurement of bioelectrical activity are the high-density multichannel thin film iEMG electrodes. Electroencephalography (5) (EEG) sensors are also used and are responsible for detecting the user's voluntary intention to move.

In the example, implantable thin film electrodes (8) are used for neurostimulation and are responsible for the generation of electric fields that modulate the upper limb afferent pathways, projecting the afferent inflow to spinal and supraspinal centers, thereby causing the interruption of the low frequency synchronization in corticobasal ganglia circuits and, consequently, the suppression of tremor.

All algorithms used for cancellation of tremor could be implemented using programming environments that are suitable for the development of real time systems (e.g., C++). The algorithms are implemented in a microcontroller (integrated in the programmable electronic device (9)) capable of capturing all the sensor signals. In this particular example the acquisition rate is 200 kHz and the resolution 12 bits. This microcontroller also incorporates a Bluetooth module for wireless communication with the external device (2), not shown in the Figure.

The strategy for identifying and monitoring the tremor is based on the signals from the sensors (bioelectrical (3) and biomechanical (4)). The EEG signal sensors provide the information about the patient intent to move voluntarily by integrating two Bayesian classifiers that are based on different characteristics of the event related desynchronization (ERD) in EEG signals. EMG sensors indicate the onset of tremor and finally, the amplitude and frequency of the tremor are extracted from the inertial sensor data. The sampling frequency chosen for the loop identification is 1 KHz.

As external device (2) responsible for the implementation of the user interface is based on an iPad. The user interface is programmed into the iOS operating system. The communication between the programmable electronic device (9) and the external device (2) is made via Bluetooth.

The platform in this example provides long-term records of neural and biomechanical data of upper limb tremor. The tremor is treated during the patient's daily life by afferent nerve stimulation. The collected information is analyzed, and metrics are derived in order to provide the feedback about the performance of the therapy to the patient and the clinician. The remote medical professional has remote access to these data via the interface programmed in the iPad, and is able to adjust the therapy. As a result, the user is more involved in the treatment.

The final product will thus be an aesthetically appealing wearable neuroprosthesis that will integrate all the electronics for control, acquisition and processing, and the flexible inertial sensors on a textile substrate, together with a set of implantable thin film electrodes for neurostimulation and neural recordings. The neuroprosthesis constitutes an alternative management of tremor, either alone or in combination with pharmacotherapy.

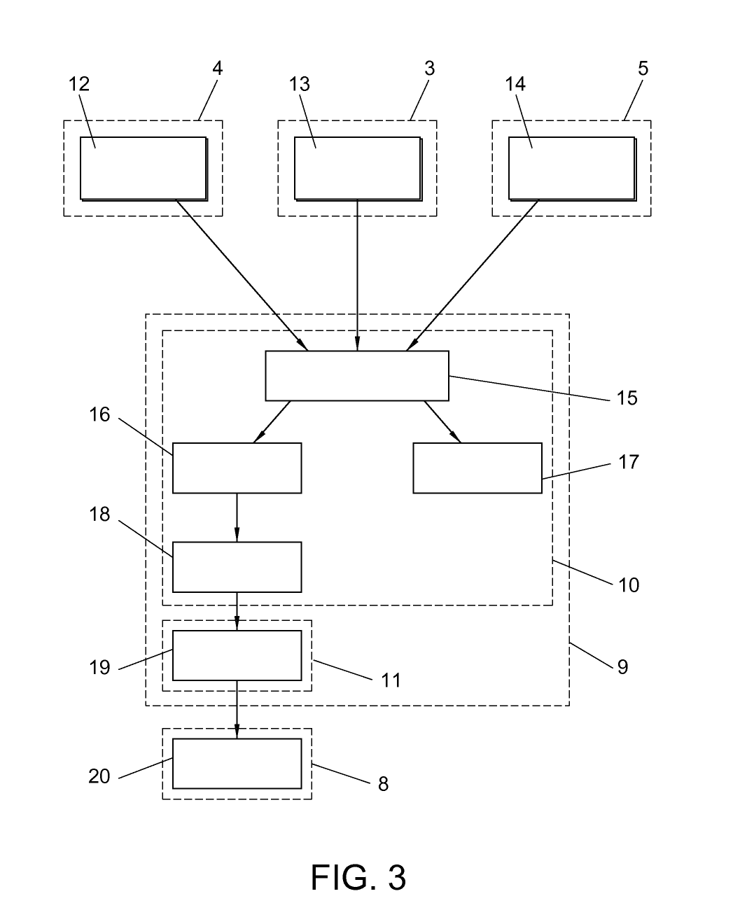

FIG. 3 shows a particular example of the method for monitoring and suppression of pathological tremors in which biomechanical characterization signals (12), captured by the inertial sensors (4), bioelectrical characterization signals (13), captured by the electromyography sensors (3) and electroencephalography sensors (14) are sent to the programmable electronic device (9) which differentiate (15) the tremor (16) from the voluntary movement (17) of the user. This operation is performed in the control acquisition and processing module (10) of the electronic device (9). From the characterization of tremor signal (16) the control module (10) calculates (18) the necessary neurostimulation to compensate the tremor (16). This signal is sent to neurostimulation signal generator (11), generating (19) this signal and sending it to the neurostimulation electrodes (8) that eventually generate (20) the afferent inflow to the central nervous system.

### Example 1

In this example we present one particular implementation of the method and device object of this invention that was tested in patients as a proof-of-the-concept. The tests demonstrated that indeed afferent stimulation can be used to suppress tremor.

The device estimates tremor from the recorded EMG signals online by using an algorithm based on the Iterative Hilbert Transform (IHT) that we described previously in an offline implementation (J. L. Dideriksen et al. (2011) EMG-Based Characterization of Pathological Tremor Using the Iterated Hilbert Transform, IEEE Transactions on Bio-Medical Engineering, vol. 58, no. 10, pp. 2911-2921). Stimulation was delivered over the motor points of the wrist and finger flexors and extensors out of phase with respect to the tremulous EMG activity. The device was tested in 6 tremor patients and the results with afferent stimulation were compared to those obtained in the same patients using the stimulation above the motor threshold (motor stimulation), considered as the reference standard (classical approach).

### A. Device Components (Hardware)

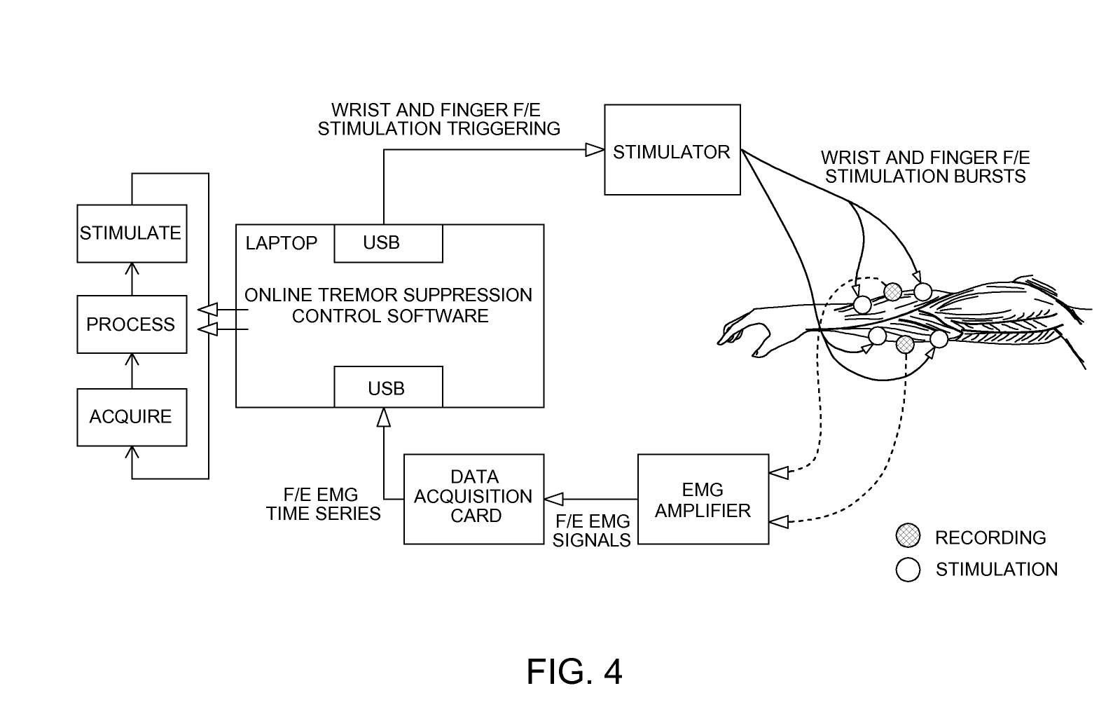

The neuroprosthetic device comprised the following components (FIG. 4): 1) Bioelectrical sensor i.e., an EMG amplifier (AnEMG12, OTBioelettronica, IT) with two analog bipolar channels and standard ø11 mm Ag/AgCl electrodes (Neuroline 720, Ambu, DK), 2) Programmable electronic device with processing module (standard laptop, dual core at 2 GHz), acquisition module (NI USB 6210, National Instruments, US), and electric signal generator, i.e., stimulator (TremUNA, UNA Systems, SR). Bioelectric sensors were used to detect EMG from the wrist and finger flexors and extensors. The amplifier channels with adjustable gains were equipped with analog band-pass filters with the cut-off frequencies at 10 and 500 Hz. The EMG signals were sampled at 1 kHz by the data acquisition card connected via a USB port to the laptop. The laptop ran custom-made software for tremor suppression, which controlled the acquisition, processed the EMG data and commanded the stimulation unit, as described in “B. Device operation (see below)”. The stimulator was battery powered, current-controlled and fully programmable; it was connected to the laptop via USB, through which it received textual commands allowing the online control of the stimulation timing and intensity. The USB communication components were optically isolated from the rest of the stimulator electronics. Four stimulation channels in monopolar electrode configuration (common anode) were used to activate wrist and finger flexor and extensor muscles. Disposable square (5 cm×5 cm) and round (ø3.2 cm) electrodes (PALS Platinum, Axelgaard, US) were used for the stimulation of the wrist flexors/extensors and finger flexors/extensors, respectively. Therefore, the device was suitable for the suppression of the flexion/extension tremor.

### B. Device Operation

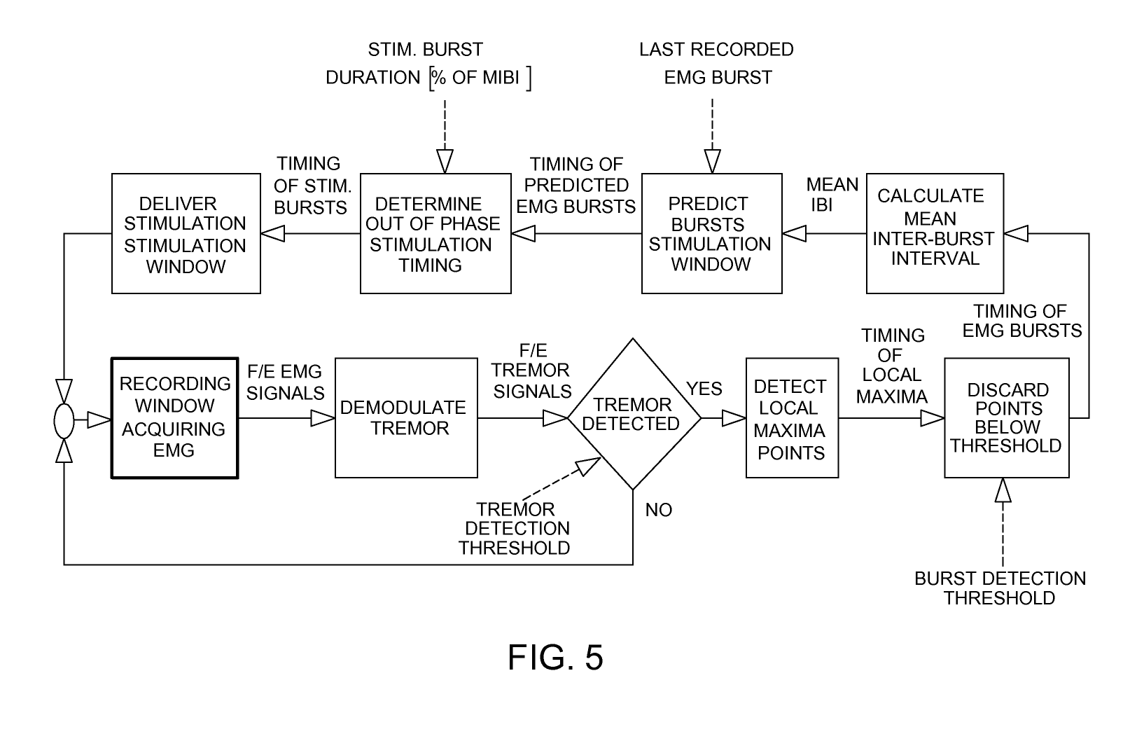

The device implemented a sequential strategy, interleaving recording and stimulation periods (FIGS. 4 and 5), i.e., the EMG was captured during the recording time window, the data were processed to determine tremor parameters and stimulation timing, and the stimulation was delivered during the stimulation time interval. This strategy was adopted in order to avoid the presence of stimulation artifacts in the EMG signals. The durations of the time windows can be easily adjusted by the skilled person. The aforementioned steps (i.e., recording, processing, and stimulation) were repeated in a loop.

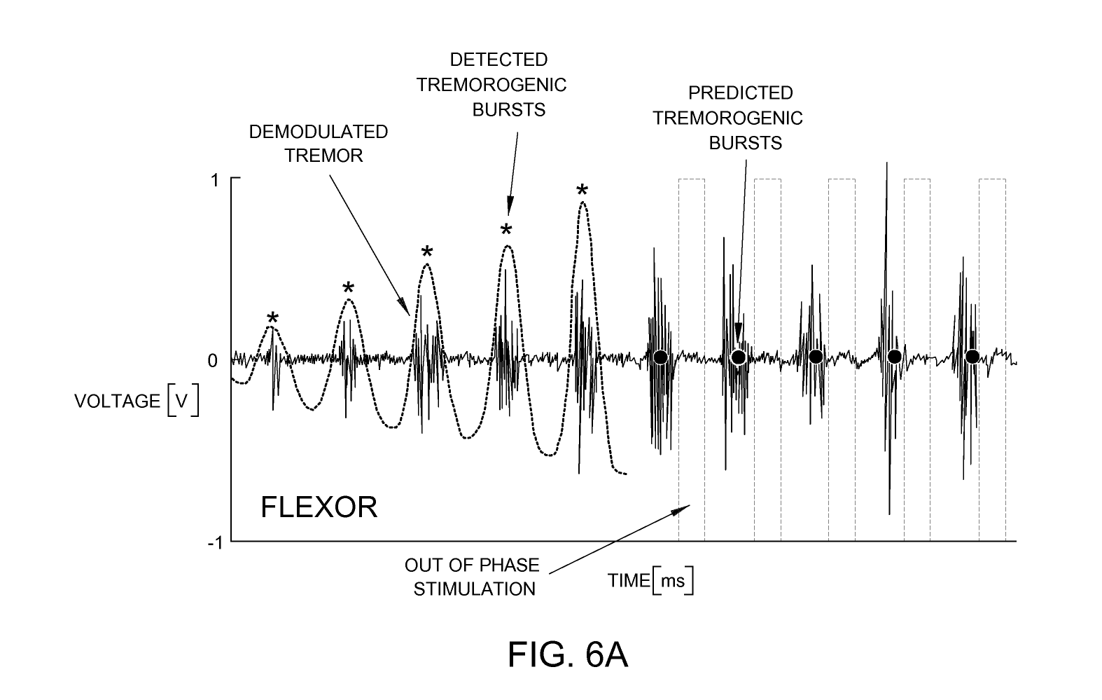

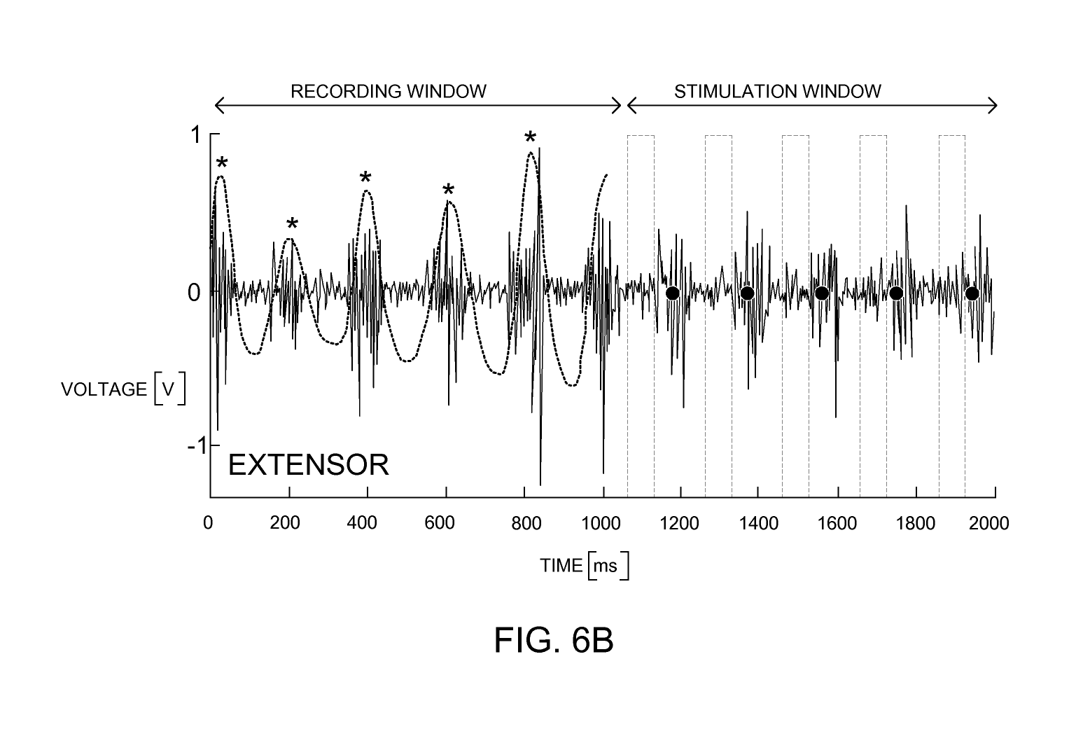

To estimate tremor parameters, we implemented online our recently developed method for tremor demodulation based on the Iterative Hilbert Transform (IHT) (J. L. Dideriksen et al. (2011) EMG-Based Characterization of Pathological Tremor Using the Iterated Hilbert Transform, IEEE Transactions on Bio-Medical Engineering, vol. 58, no. 10, pp. 2911-2921). The IHT was applied to the rectified EMG signal, and the first IHT component band-passed between 3 and 12 Hz was an estimate of the tremorogenic EMG activity (separated from the voluntary component). If the root mean square of the demodulated tremor signal (FIG. 5) was above a user defined threshold (i.e., tremor detection threshold), the tremor was deemed to be present. If the tremor was detected, the centers of tremorogenic EMG bursts were determined by finding the local maxima of the tremor signal. Only the maxima that were above the burst detection threshold were taken into account. The thresholds for tremor and burst detection were selected by the skilled person. The mean inter-burst interval (MIBI) was then calculated, and the subsequent tremorogenic bursts expected to appear within the stimulation window were predicted based on the calculated MIBI and the timing of the last recorded burst. This processing was performed for each muscle group (flexor/extensor) independently. The stimulation was delivered out of phase with the estimated tremorogenic activity. The duration of the stimulation bursts was defined manually, by the skilled person, as a percentage of the MIBI, and the stimulation burst delivered to the flexor (extensor) muscle coincided with the center of the predicted tremorogenic EMG burst of its antagonist, i.e., the extensor (flexor) muscle (out of phase stimulation). The stimulation timing took into account the delays due to signal acquisition and processing. One example of the processing is given in FIG. 6A and FIG. 6B. In this particular example, the recording and stimulation windows were 1 second long. FIG. 6A and FIG. 6B show the flexor and extensor EMG, respectively, and the dashed line is the estimated tremor demodulated from the EMG. The stars denote the estimated centers of the tremorogenic bursts for the EMG captured during the recording window (tremor signal maxima), and the full black circles are the predicted centers of the tremorogenic bursts within the stimulation window. The dashed bars denote the time intervals during which the stimulation bursts are delivered. The occurrences of the predicted bursts match very well those of the actual bursts within the stimulation window, and the stimulation was delivered out of phase with tremorogenic activation.

### C. Online Control Software

The software for online configuration and control of the neuroprosthetic device was developed in Visual C# (Visual Studio, Microsoft, US), using Measurement Studio controls (National Instruments, US), and implemented a user friendly interface for online device monitoring and setup. The main window comprised a Recording and Stimulation tab. The Recording tab included the fields for setting up the parameters controlling the EMG acquisition (e.g., sampling rate), tremor demodulation (i.e., the thresholds for tremor and burst detection) and device operation (e.g., duration of the recording and stimulation/prediction windows, stimulation burst duration in percent of MIBI). The Stimulation tab was used to select the stimulation channels, adjust the parameters (frequency, pulse width and amplitude) using sliders, and manually start and stop the stimulation on the specific channel (e.g., for testing the motor points). The device has two operating modes: monitoring and tremor suppression. In the former, the system records the data, displays the processed signals to the experimenter, and depicts the stimulation bursts graphically but without delivering the stimulation. This

TABLE I
RECRUITED PATIENTS

| | | | | | | Tremor | |
|---|---|---|---|---|---|---|---|
| Patient | | Age | | | Tremor | Frequency | Tremor |
| ID | Gender | [years] | UPDRSII | FTMS | severity | [Hz] | etiology |
| PD1 | Male | 75 | 21 | — | Mild (1) | 3.9 | Parkinson |
| | | | | | | | disease |
| PD2 | Male | 58 | 20 | — | Severe | 3.3 | Parkinson |
| | | | | | (3) | | disease |
| PD3 | Female | 76 | 23 | — | Severe | 4.2 | Parkinson |
| | | | | | (3) | | disease |
| PD4 | Male | 67 | 18 | — | Moderate | 5.2 | Parkinson |
| | | | | | (2) | | disease |
| ET1 | Female | 73 | — | 49 | Moderate | 4.7 | Essential |
| | | | | | (2-3) | | tremor |
| ET2 | Male | 71 | — | 34 | Moderate | 4.5 | Essential |
| | | | | | (2) | | tremor |

Notation:
UPDRSII stands for Motor Section II of the Unified Parkinson Disease Rating Scale;
FTMS is Fahn-Tolosa-Marin tremor scale;
Tremor severity was evaluated using the Whiget tremor scale.

Parkinson disease (PD). Inclusion criteria included predominant flexion/extension tremor at the wrist. The characteristics of the recruited patients are reported in Table I. The experiments were conducted in accordance with the Declaration of Helsinki and approved by the local ethical committee. All participants signed a written informed consent before the inclusion.

### E. Experimental Protocol

The patients were comfortably seated in a chair in front of a desk. In the case of bilateral tremor, the neuroprosthetic system was applied to the more affected side. The dorsal and volar sides of the forearm were gently cleaned with a wet sponge. The muscle motor points were identified by using a probing electrode (i.e., stimulation electrode with a wet sponge on the top). The electrode was initially placed as recommended in (L. L. Baker, et al. (2000) Neuro Muscular Electrical Stimulation: A Practical Guide (4th Edition), 4th ed. Los Amigos Research Institute, Inc.) for the stimulation of the wrist and finger flexors and extensors, and then moved around in small steps, until the point resulting with the strongest motor response was identified. During the probing, the stimulation frequency was set to 100 Hz, the pulse width to 300 us, and the current amplitude to a comfortable level producing motor response (flexion/extension of the wrist/fingers). After the electrode location was determined, the self-adhesive stimulation electrodes were placed over the motor points. Stimulation frequency and current pulse width were set to 100 Hz and 300 us, and the pulse amplitude was adjusted to obtain a flexion or extension of the joint. As muscle contractions induced by electrical stimulation over the muscle belly are known to be almost exclusively mediated by motor axon recruitment (A. J. Bergquist et al. (2012) Motor unit recruitment when neuromuscular electrical stimulation is applied over a nerve trunk compared with a muscle belly: quadriceps femoris, Journal of applied physiology (Bethesda, Md.: 1985), vol. 113, no. 1, pp. 78-89; A. J. Bergquist et al. (2011) Motor unit recruitment when neuromuscular electrical stimulation is applied over a nerve trunk compared with a muscle belly: triceps surae, Journal of applied physiology (Bethesda, Md.: 1985), vol. 110, no. 3, pp. 627-37) this was referred to as the motor stimulation. To activate selectively the sensory fibers, the current was decreased below the motor threshold (afferent stimulation). The electrodes placed over the finger muscles were used only during the afferent stimulation in order to increase the recruitment of the sensory fibers. Absence of motor response during the sensory stimulation was verified visually and by palpating muscles and tendons. This was done with one (e.g., wrist flexor) and two channels (e.g., wrist and finger flexor) active, to check for the possible “summation” of the charge in the tissue due to the simultaneous activation of the channels. The neutral electrode (5×9 cm) was placed at the wrist.

The skin was prepared for the EMG recordings by gently applying a small amount of abrasive paste. The electrodes were placed on the dorsal and volar sides of the forearm approximately half way between the elbow and the wrist. The ground band (circumferential electrode) was secured around the wrist joint. The amplifier gain was increased until the tremor EMG bursts became clearly visible. The recording and stimulation windows were set to 1 and 3 s as in (L. Popovie Maneski et al. (2011) Electrical stimulation for the suppression of pathological tremor,” Medical & Biological Engineering & Computing, vol. 49, no. 10, pp. 1187-1193), respectively.

Tremor and burst detection thresholds were set initially to 0.25 V and 0.05 V, which are the values found in pilot tests. The tremor control software was then used in the monitoring mode to assess if the system detected tremor correctly and consistently in a specific patient, and this was checked visually, by comparing the onset of tremor in the patient and the corresponding software output (detected and demodulated tremor). If necessary, the threshold parameters were further adjusted in order to improve the tremor detection. The duration of the stimulation burst was set to 40% of the MIBI, since this value allowed several pulses to be delivered within a stimulation burst while also assuring that the bursts were well separated. The average tremor frequency in this study was 4 Hz, which means that approximately 10 pulses were delivered per stimulation burst.

To assess tremulous movements, we used inertial measurement system (XBus kit, XSens, NE) comprising a control box and a set of inertial units (MTx, XSens, NE) measuring the full 3D orientation of the segments to which they were attached. Two units were strapped to the dorsal sides of the forearm and hand so that the wrist flexion/extension joint angle could be computed. The system performance was assessed by comparing the power of the tremor signals (wrist angle) as measured by the inertial measurement units during the intervals with the device on vs. intervals with the device off (baseline).

Postural tremor is characteristic of Essential tremor (ET), and therefore the task for ET patients was to hold the arm outstretched unsupported against gravity (G. Deuschl et al. (1998) Consensus statement of the Movement Disorder Society on Tremor. Ad Hoc Scientific Committee., Movement disorders, vol. 13 Suppl 3, pp. 2-23). Parkinson disease patients exhibit tremor mainly at rest, and therefore, these subjects were instructed to position the forearm on a convenient support and relax (G. Deuschl, P. et al., cited above). In addition, the patients were sometimes engaged in distracting tasks (e.g., speaking, counting backwards, drawing shapes with contralateral hand), since this is known to trigger and/or worsen the tremor.

To prevent fatigue due to keeping the posture and/or stimulation of the muscles at the motor level, the recordings included trials of approximately 2 min each. Patients were allowed to rest between the trials for a time period under their discretion. The first two trials were used to record the tremor exhibited by the patient while the device was off (baseline tremor, “DEVICE OFF” condition). Only the portions of the signals with a clearly visible tremor (wrist angle oscillations >2°) were considered. Further trials served to assess the performance of tremor suppression using motor and afferent level stimulation. Within each trial, the inertial measurement system was started first, and once the tremor had developed at approximately the same level as during the baseline recordings, the neuroprosthetic device was turned on. While the device was active, the electrical stimulation was delivered intermittently, i.e., the recording and stimulation were implemented sequentially (as explained in B. “Device Operation”, see above). Afferent stimulation was tested first in order to introduce the subjects to the sensation of electrical stimulation gradually, starting with the low intensities. It is known that tremor is very variable phenomena, and it was sometimes difficult to evoke a consistent tremor in the patients. However, at least five trials of tremor suppression have been recorded for each modality. In total, the measurement session lasted for approximately 2.5 h.

### F. Data Analysis

To evaluate the device performance, the segments during which the device was active were extracted from each device test trial and further separated into recording and stimulation intervals. We have compared the following conditions: 1) “DEVICE ON” vs. “DEVICE OFF”, 2) “DEVICE ON (STIMULATING)” vs. “DEVICE OFF”, 3) “DEVICE ON (RECORDING)” vs. “DEVICE OFF”, and 4) “DEVICE ON (STIMULATING)” vs. “DEVICE ON (RECORDING)”. The goal of the first comparison was to evaluate the amount of tremor suppression while the device was active, sequentially recording and stimulating, with respect to the baseline tremor (device inactive). The second and third comparison assessed the level of tremor suppression separately within the stimulation and recording intervals, respectively. Finally, the two intervals (recording and stimulation) were compared to each other. We concatenated all the signal segments from one condition, computed the periodogram of the resulting waveform and calculated the tremor power by integrating over the tremor frequencies (between 3 and 12 Hz). The average percent suppression was defined as 100%*(1−PR), where the PR (power ratio) is the ratio of the tremor powers in the compared conditions.

One-way repeated measures ANOVA with the stimulation condition as within subjects factor was used to test for the statistically significant difference in the tremor power. In total, there were 7 conditions, i.e., [SENSORY, MOTOR]×[ON, STIMULATION, RECORDING], and SYSTEM OFF. Post hoc analysis (Newman-Keuls test) was applied for the pairwise comparisons. The threshold was adopted at p<0.05.

### Results

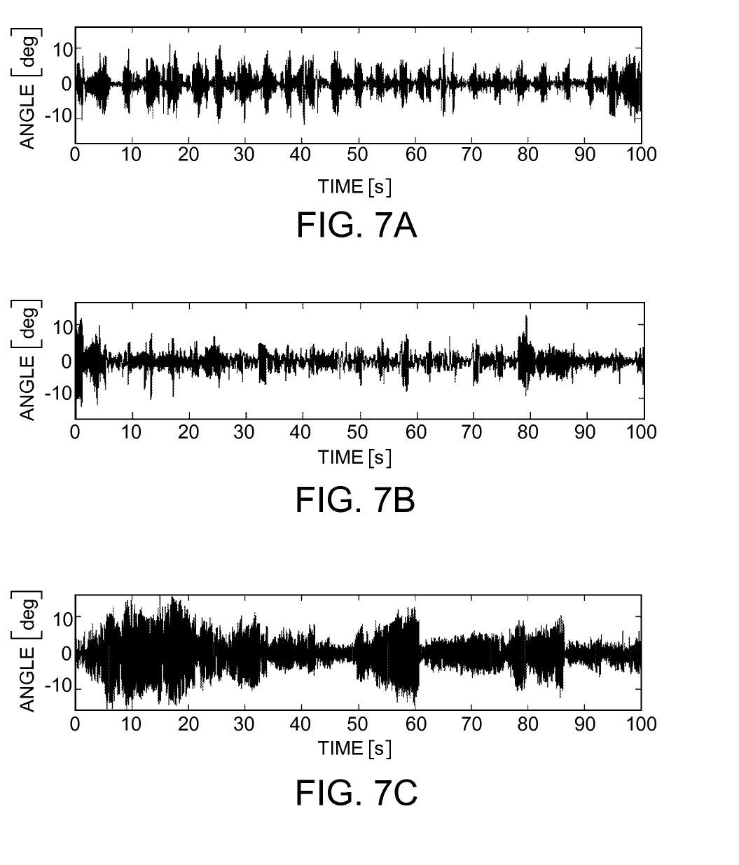

As representative case, we first illustrate the performance of the neuroprosthetic device by presenting detailed results for one patient (ET1). FIGS. 7A to 7C depicts the wrist flexion/extension joint angle recorded by the inertial sensors while the device was off (FIG. 7C) and while it was active (FIG. 7A, FIG. 7B). In the latter case, the device delivered stimulation intermittently, between the recording periods, either below (afferent stimulation, FIG. 7A) or above (motor stimulation, FIG. 7B) the motor threshold (as described in E. “Experimental protocol”, see above). The figure depicts long signals generated by concatenating several trials and provides a global insight into the operation and impact of the neuroprosthetic device. While the device was off, tremor oscillations were continuous and pronounced, although with a somewhat variable amplitude (peak-to-peak amplitudes between 5° and 25°). Suppression was evident during both motor and afferent stimulation, although the tremor was more attenuated during the motor stimulation.

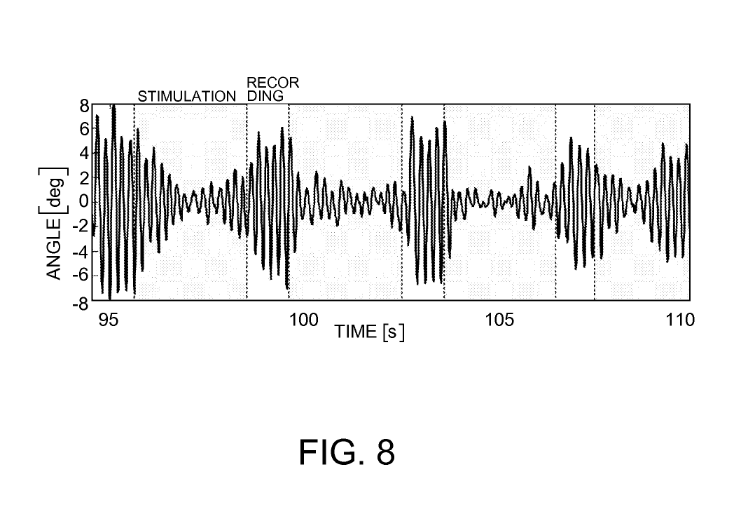

As it is seen in FIGS. 7A to 7C, the recorded signals exhibited a characteristic structure during the device operation, i.e., the traces in FIGS. 7A to 7C are comprised of interleaved segments of higher and lower amplitude wrist oscillations. This reflected a specific pattern of tremor attenuation due to the adopted sequential strategy: tremor was clearly suppressed while the stimulation was being delivered, but then it would recover as the device was recording without stimulating. FIG. 8 depicts the wrist flexion/extension joint angle during the recording and stimulation time intervals within a segment of the trial in which the device was active delivering the stimulation below the motor threshold (afferent stimulation). Tremor was suppressed during the stimulation with respect to recording intervals but the performance was inconsistent. Sometimes tremor was attenuated sharply with the transition between the recording and stimulation, and the attenuation was stable throughout the whole stimulation period (FIG. 8, 2nd and 3rd stimulation interval). Yet, in the very next interval, the amplitude would gradually decrease at the beginning and/or recover to a certain extent towards the end of the stimulation (FIG. 8, 1st and 4th stimulation interval).

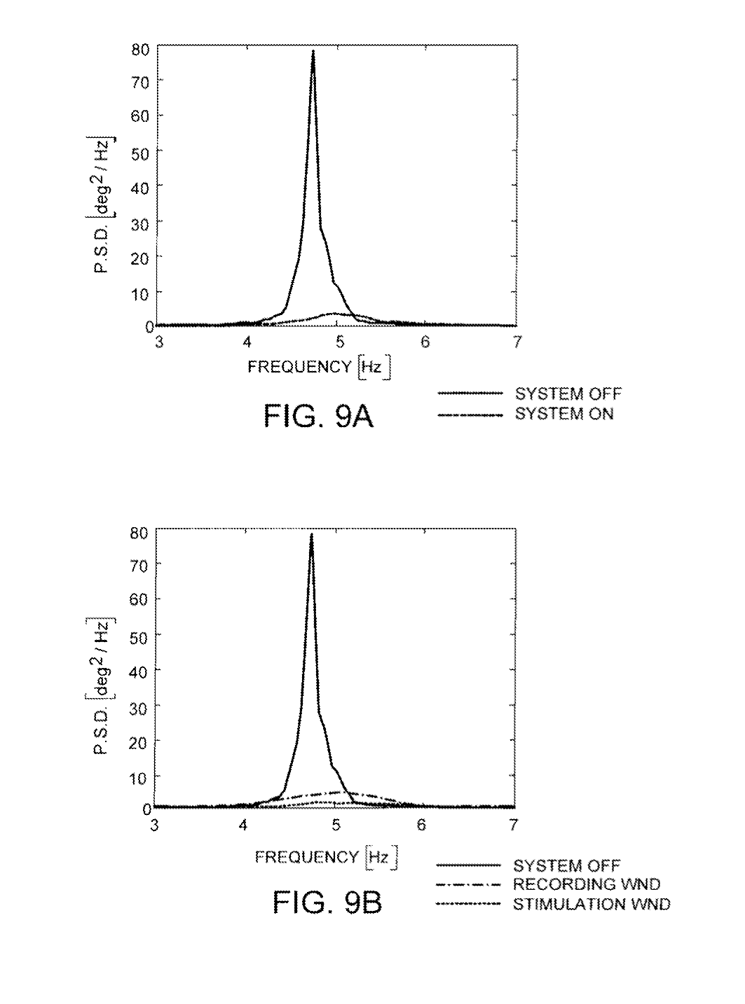

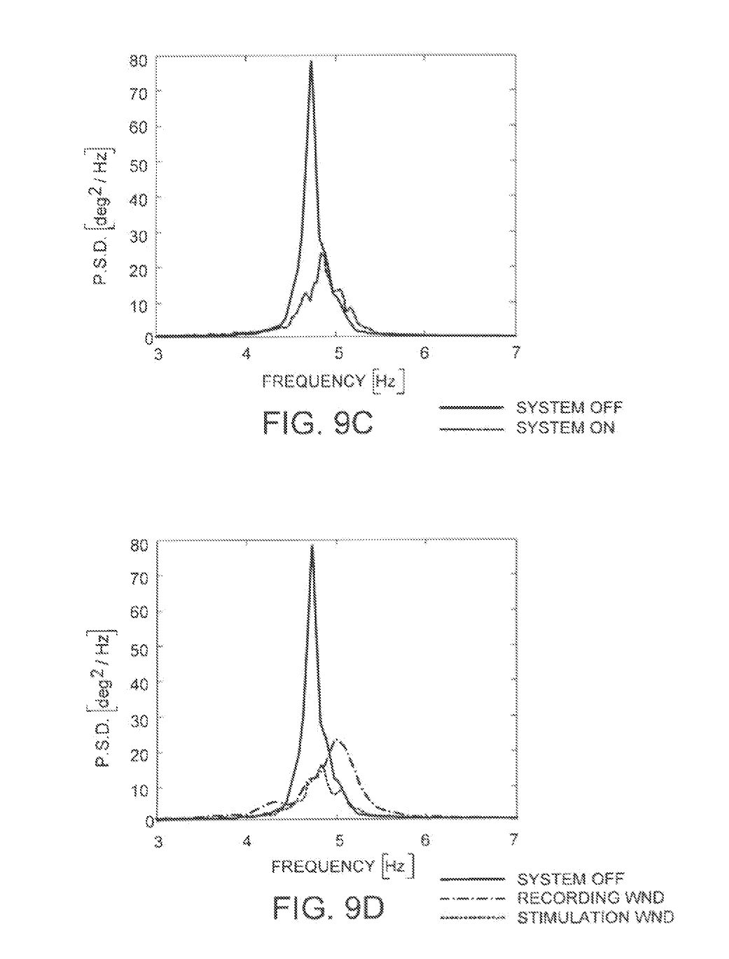

Frequency analysis (FIGS. 9A to 9D) quantitatively confirmed the qualitative conclusions from FIGS. 7A to 7C and 8. The drop in the signal power was most pronounced while the device was actually delivering the stimulation (FIG. 9B, FIG. 9D). Importantly, although the tremor was re-emerging between the stimulations (i.e., in the recording intervals), it did not recover to the full extent: the average tremor power was lower with respect to the baseline (device off) even when considering only the intervals during which the device was passively recording without stimulating. As a result, there was an overall decrease in the signal power while the device was active, i.e., stimulation and recording taken together vs. device off (FIG. 9A, FIG. 9C). Motor stimulation was more successful in suppressing tremor both during

TABLE II
PERCENT TREMOR SUPPRESSION

| | | | DEVICE ON | | |
|---|---|---|---|---|---|
| | | DEVICE | (STIMULATING) | DEVICE ON | DEVICE ON |
| | | ON vs. | vs. DEVICE ON | (RECORDING) | (STIMULATING) |
| Patient | Stimulation | DEVICE | (RECORDING) | vs DEVICE | vs. DEVICE |
| ID | modality | OFF [%] | [%] | OFF [%] | OFF [%] |
| PD1 | Motor | 46 | 64 | 4 | 66 |
| | Afferent | 48 | 37 | 32 | 57 |
| PD2 | Motor | 52 | 58 | 23 | 67 |
| | Afferent | 35 | 73 | −29 | 65 |
| PD3 | Motor | 53 | 71 | 10 | 74 |
| | Afferent | 44 | 45 | 20 | 56 |
| PD4 | Motor | 68 | 60 | 42 | 77 |
| | Afferent | 41 | 35 | 21 | 49 |
| ET1 | Motor | 81 | 61 | 67 | 87 |
| | Afferent | 43 | 49 | 13 | 56 |
| AVERAGE | Motor | 60 ± 14 (**) | 63 ± 5 (*) | 30 ± 25 | 74 ± 8 (**) |
| | Afferent | 42 ± 5 (*) | 48 ± 15 | 12 ± 24 | 57 ± 6 (*) |

Notation: the star denotes the statistically significant difference (*, p < 0.05; **, p < 0.01).

stimulation and recording intervals.

### Overall Results

The levels of tremor suppression for all patients are shown in Table II.

We could not suppress tremor in the patient ET2 with either of the two stimulation modalities. This subject was treated as a special case and was therefore excluded from the comparison. Importantly, the fact that the device failed to suppress tremor in this case is not due to etiology, since good results were obtained in the second patient with Essential tremor (ET1). The current experiment was performed with a predefined parameters (e.g., stimulation frequency and amplitude) in order to have a consistent comparison between subjects and conditions. The fact that these parameters were not tuned could be the reason for the lack of suppression in this particular subject.

While the device was active (stimulation and recording), the average tremor suppression mean±standard deviation) was 60±14% and 42±5% for above (motor stimulation) and below (afferent stimulation) motor threshold stimulation, respectively. Both operation modes decreased the power of tremor signal significantly with respect to the baseline (p<0.01 for motor and p<0.05 for afferent stimulation). Importantly, if only the stimulation intervals were considered, the average suppression for motor and afferent stimulation increased to 74±8% and 57±6%, respectively (p<0.01 for motor and p<0.05 for afferent stimulation with respect to the baseline). Tremor was attenuated more during the stimulation than during the recording (p<0.05), but this was statistically significant only for the motor stimulation. If the recording intervals were considered alone, the average results suggested that there was a certain level of suppression with respect to the baseline, but the difference was not statistically significant, likely due to the small number of subjects. For one subject (PD2), when the stimulation was delivered below the motor threshold (afferent stimulation), the average tremor power during the recording was higher than while the device was off. Finally, although the stimulation above the motor threshold (motor stimulation) outperformed the afferent stimulation on average, the statistical tests showed no significant differences between the two. Again, the likely reason for the lack of significance is the small number of subjects. The average stimulation levels were 15±3 mA and 18±5 mA for the wrist flexor and wrist extensor in the motor stimulation versus 9±3 mA, 6±2 mA, 10±3 mA and 6±2 mA for the wrist and finger flexors, and wrist and finger extensors in the afferent stimulation.

Therefore, we presented here a preferred neuroprosthetic device, which uses a method based on the IHT to estimate tremorogenic EMG activity from the EMG acquired during the recording window, predicts the tremorogenic bursts that will occur within the subsequent stimulation window, and then delivers the stimulation to the antagonistic muscle pair according to this prediction. The control software was developed using a state of the art application development environment (Visual C#) for good online performance, and also integrates a user friendly graphical interface suitable for the prospective clinical use. We tested the device in 6 tremor patients by using two stimulation modalities: above the motor threshold (classic approach) and below the motor threshold (novel approach).

The tests demonstrated that the neuroprosthetic device was able to substantially attenuate tremor in both stimulation modalities. The suppression while the device was active (recording and stimulation windows) and while the stimulation was being delivered (stimulation windows) was within the values reported previously for motor stimulation (73% and 62% (A. Prochazka et al. (1992) Attenuation of pathological tremors by functional electrical stimulation. I: Method, Annals of Biomedical Engineering, vol. 20, no. 2, pp. 205-224; M. Javidan et al. (1992) Attenuation of pathological tremors by functional electrical stimulation. II: Clinical evaluation, Annals of Biomedical Engineering, vol. 20, no. 2, pp. 225-236), 67% (L. Popović Maneski et al. (2011) Electrical stimulation for the suppression of pathological tremor, Medical & Biological Engineering & Computing, vol. 49, no. 10, pp. 1187-1193), 52% (J. A. Gallego et al. (2013) A neuroprosthesis for tremor management through the control of muscle co-contraction, Journal of neuroengineering and rehabilitation, vol. 10, p. 36), and 57% (F. Widjaja et al. (2011) Sensing of pathological tremor using surface electromyography and accelerometer for real-time attenuation, Journal of Mechanics in Medicine and Biology, vol. 11, no. 05, pp. 1347-1371)). The amount of tremor attenuation in the current implementation was limited by the sequential operation of the tremor detection and suppression loops. This strategy was adopted in order to avoid the stimulation artifacts in the EMG. However, with a more sophisticated setup, including an amplifier with a blanking input and a suitable artifact suppression method, the recording, processing and stimulation can be performed concurrently. This allows the device to reach its full potential and achieve a higher level of tremor suppression consistently and continuously throughout its operation. Furthermore, the suppression performance may be further enhanced by optimization of the stimulation parameters. To limit the duration of the experimental session for the elderly patients, pulse width and stimulation frequency, known to have an effect on afferent stimulation efficiency (O. Lagerquist et al. (2010) Influence of stimulus pulse width on M-waves, H-reflexes, and torque during tetanic low-intensity neuromuscular stimulation, Muscle & nerve, vol. 42, no. 6, pp. 886-93; J. M. Clair et al. (2011) Postactivation depression and recovery of reflex transmission during repetitive electrical stimulation of the human tibial nerve, Journal of neurophysiology, vol. 106, no. 1, pp. 184-92) were not systematically varied.

In most of the closed loop systems for counteracting tremor based on electrical stimulation, the stimulation was controlled by means of a signal from inertial units. In the preferred neuroprosthetic device described in this patent application, tremor detection was based on EMG, according to an algorithm we previously proposed and tested offline (J. L. Dideriksen et al. (2011) EMG-Based Characterization of Pathological Tremor Using the Iterated Hilbert Transform, IEEE Transactions on Bio-Medical Engineering, vol. 58, no. 10, pp. 2911-2921). As demonstrated in this Example 1, the algorithm can be used for online detection and demodulation of tremor. The preferred neuroprosthetic device used in this Example has been tested in a larger population of patients with tremor of different etiologies. The tests showed that the results are generalizable as far as the muscles responsible for tremor are superficial. An important feature of this preferred device is that it can separate the tremulous from the voluntary component of EMG activation. Therefore, the device stimulates only in the presence of tremor and the stimulation time is minimized, lowering the discomfort for the patient and increasing the device battery lifetime.

Thus, this invention provides a method and neuroprostetic device that represents a first demonstration that tremor suppression may be achieved by delivering electrical stimulation below the motor threshold (afferent stimulation), stimulating thereby mainly the sensory fibers. Using afferent stimulation, the tremor is suppressed and the average attenuation degree is not substantially different than with the classic motor approach. The stimulation frequency was higher (@100 Hz) compared to what is usually applied in FES but it was within the range used in neuromodulation applications targeting the sensory pathways (e.g., pain suppression) (P. Hansson et al. (1983) Transcutaneous electrical nerve stimulation (TENS) as compared to placebo TENS for the relief of acute oro-facial pain, Pain, vol. 15, no. 2, pp. 157-65). It is known that a prolonged higher level motor stimulation at the same frequency would produce muscle fatigue (L. L. Baker et al. (2000) Neuro Muscular Electrical Stimulation: A Practical Guide (4th Edition), 4th ed. Los Amigos Research Institute, Inc.), imposing the necessity of increasing the intensity of current delivered. On the other hand, muscle fatigue is not a constraint for the proposed sensory stimulation due to a lack of motor response. Since tremorogenic EMG bursts can be quite short depending on the tremor frequency, we opted for the high frequency stimulation in order to be able to deliver more pulses within each burst, thereby providing a stronger afferent input.

The method for the monitoring and suppression of pathological tremor via sensory stimulation, as described in the present invention presents several advantages over previous methods. The fact that the stimulation may be effective even when delivered below the motor threshold avoids fatigue and potential pain due to high stimulation intensities typical of other devices. This is even more important in case of elderly patients, who are the majority in the tremor population. The advantages can be most noticeable when the patient is performing daily living activities as sensory stimulation attenuates tremor without interfering with functional movements, contrary to motor stimulation. The neuroprosthetic device of the invention can be thus more comfortable for the patient and more effective in restoring daily task functionality.

The proposed device requires minimal hardware that is easily scalable. In our experiments, we targeted the wrist joint. However, the device can be applied simultaneously to the two joints (e.g., wrist and elbow), as the stimulator features 8 channels and the EMG amplifier 12 channels. The use of EMG as a control signal to drive tremor suppression potentially holds several advantages compared to inertial sensors. First, it allows tremor detection from the muscles directly responsible for the mechanical oscillation (“cause”) rather than from the oscillation signal itself (“effect”). Contrary to devices that are based on inertial measurement units for detecting tremor and triggering stimulation (J. A. Gallego et at. (January 2013) A neuroprosthesis for tremor management through the control of muscle co-contraction, Journal of neuroengineering and rehabilitation, vol. 10, p. 36), in the proposed technique tremorogenic activity is estimated at the level of individual muscles/muscle groups, which implies that the corrective action (stimulation) can be tailored to each muscle/muscle group independently for optimizing the tremor compensation. We have indeed observed that tremor can be produced by only one muscle or that different muscles can be producing tremor depending on the current activity of the subject. In these cases, a selective activation might be of interest and a unique feature of the method could be fully exploited by developing an implantable system that would allow very specific activation of sensory-motor structures. Moreover, sensors for EMG detection are placed more proximally than inertial units and are therefore more discrete and less visible, which could improve the cosmetics of the potential future orthotic system.

In its current implementation, the device is designed for the monitoring and suppression of pathological tremor such as flexion/extension tremor. However, tremor may occur at multiple degrees of freedom in a joint. For instance, pronation-supination tremor is a typical feature of Parkinson disease patients (M. Manto et al. (2008) Tremor: from pathogenesis to treatment. San Rafael, Calif.: Morgan & Claypool; G. Deuschl et al. (1998) Consensus statement of the Movement Disorder Society on Tremor. Ad Hoc Scientific Committee, Movement disorders, vol. 13 Suppl 3, pp. 2-23). The muscles responsible for those movements are deep and therefore not accessible with surface electrodes. This problem may be solved using intramuscular electrodes for recording/stimulation.

In conclusion, a neuroprosthetic device for monitoring and suppression of pathological tremor using electrical stimulation of sensory nerves based on EMG recordings was developed and tested on 6 tremor patients. The level of suppression varied across subjects as well as across trials within each patient, but was on average 42% (recording and stimulation) and 57% (stimulation only), indicating the availability of the proposed method for implementation in tremor suppression devices. This is the first proof of concept of a peripheral stimulation method for tremor suppression at low intensity stimulation currents, eliciting mainly sensory fibers.

1. Neuroprosthetic device for monitoring and suppression of pathological tremor in a user via stimulation of the afferent pathways, characterized by comprising at least one wearable element located on the user, wherein the wearable element comprises:
At least one sensor selected from at least one bioelectrical sensor that generates a bioelectrical characterization signal of the tremor, at least one biomechanical sensor that generates a biomechanical characterization signal of the tremor and a combination thereof;
A programmable electronic device comprising at least one control, acquisition and processing module for receiving and analyzing the bioelectrical characterization signals, the biomechanical characterization signals or the combination of both characterization signals, an electric signal generator for generating a signal of the afferent nerve stimulation based on the bioelectric, biomechanical or both characterization signals defined previously and,
At least one stimulation electrode integrated in the wearable element that generates the neuromodulation of the afferent pathways.

2. Neuroprosthetic device according to claim 1, characterized by comprising at least one EEG sensor which measures brain activity and generates a electroencephalography signal which characterizes intended voluntary movement of the user with the body part suffering from tremor.

3. Neuroprosthetic device according to claim 2, characterized by comprising an external device connectable to a programmable electronic device that implements an interface for accessing the functionalities of the at least one electroencephalography sensor and the wearable element.

4. Neuroprosthetic device according to claim 1, wherein at least one stimulation electrode is an electrode selected from an implanted electrode and a transcutaneous electrode.

5. Neuroprosthetic device according to claim 1, comprising at least one bioelectrical sensor selected from sensors of conventional and high resolution electromyography, electroencephalography sensors measuring brain activity and characterizing the user intention to move or a combination of those.

6. Neuroprosthetic device according to claim 1, comprising at least one biomechanical sensor which is an inertial sensor selected from gyroscopes, accelerometers, magnetometers or a combination of those.

7. Neuroprosthetic device according to claim 5, in which the electromyography sensors are multichannel thin film electrodes.

8. Neuroprosthetic device according to claim 1, wherein at least one stimulation electrode is a thin film multi-channel electrode.

9. Neuroprosthetic device according to claim 3, wherein the programmable electronic device is selected from a computer, a smartphone and a tablet device.

10. Neuroprosthetic device according to claim 3, wherein the connection between the programmable electronic device and the external device is selected from a wireless connection or a wired connection.

11. Neuroprosthetic device according to claim 3, wherein the external device comprises means for displaying measurements taken by the neuroprosthetic diagnostic device based on the available sensors, a processor for analysis of tremor and generation of diagnostics and reporting, and a memory storage for the measurements made by the neuroprosthetic device.

12. Neuroprosthetic device according to claim 9, wherein the connection between the programmable electronic device and the external device is selected from a wireless connection or a wired connection, and the external device comprises means for displaying measurements taken by the neuroprosthetic diagnostic device based on the available sensors, a processor for analysis of tremor and generation of diagnostics and reporting, and a memory storage for the measurements made by the neuroprosthetic device.

13. Method for monitoring and suppression of pathological tremor via the stimulation of the afferent pathways to the brain of the user, making use of the device described in claim 1, characterized by comprising the following stages:
i) characterizing the tremor by means of a plurality of bioelectric sensors generating a bioelectrical characterization of the tremor, biomechanical sensors generating a biomechanical characterization signal of the tremor, or a combination thereof, wherein the bioelectrical, biomechanical or both characterization signals comprises at least the instantaneous frequency, phase or amplitude of the tremor sending these bioelectrical, biomechanical or both characterization signals to the control, acquisition and processing module of the programmable electronic device;
ii) differentiating the tremor from the voluntary movement of the user through a conventional analysis of the bioelectrical, biomechanical or both characterization signals by the control, acquisition and processing module;
iii) calculating a neurostimulation signal from the signal characterization of tremor in the control, acquisition and processing module of the programmable electronic device;
iv) generating the neurostimulation signal through the signal generator module of the programmable electronic device and,
v) To generate the neurostimulation signal to the plurality of stimulation electrodes activating the afferent pathways.

14. Method for monitoring and suppression of pathological tremors according to claim 13, characterized in that when the device has at least one EEG sensor which measures brain activity of the user, the method comprises the following steps:
i) measuring, additionally to step i) the user's brain activity by at least one EEG sensor and generating the electroencephalography signal that characterizes the intention of the user to perform a voluntary movement of the limb affected by tremor;
ii) sending, additionally in Phase II), the electroencephalography signal to the control, acquisition and processing module of the programmable electronic device;
iii) To distinguish, additionally in Phase III), the tremor from the voluntary movement of the user through a conventional analysis of the electroencephalography signal, and,
iv) To additionally employ EEG signal in step iv) to calculate the neurostimulation signal.

15. Method for monitoring and suppression of pathological tremors, according to claims 13, wherein the neurostimulation signal generate an afferent input flow to the central nervous system that disrupts the low-frequency synchronization in the corticobasal ganglia circuits.

16. Method for monitoring and suppression of pathological tremors according to claim 13, wherein the conventional analysis employed by the programmable electronic device for differentiating the tremor from the voluntary movement is based on algorithms in the time and frequency domain.

17. Method for monitoring and suppression of pathological tremors according to claim 14, wherein the neurostimulation signal generates an afferent input flow to the central nervous system that disrupts the low-frequency synchronization in the corticobasal ganglia circuits, and the conventional analysis employed by the programmable electronic device for differentiating the tremor from the voluntary movement is based on algorithms in the time and frequency domain.

18. Method for monitoring and suppression of pathological tremor via the stimulation of the afferent pathways to the brain of the user, making use of the device described in claim 2, characterized by comprising the following stages:
i) characterizing the tremor by means of a plurality of bioelectric sensors generating a bioelectrical characterization of the tremor, biomechanical sensors generating a biomechanical characterization signal of the tremor, or a combination thereof, wherein the bioelectrical, biomechanical or both characterization signals comprises at least the instantaneous frequency, phase or amplitude of the tremor sending these bioelectrical, biomechanical or both characterization signals to the control, acquisition and processing module of the programmable electronic device;
ii) differentiating the tremor from the voluntary movement of the user through a conventional analysis of the bioelectrical, biomechanical or both characterization signals by the control, acquisition and processing module;
iii) calculating a neurostimulation signal from the signal characterization of tremor in the control, acquisition and processing module of the programmable electronic device;
iv) generating the neurostimulation signal through the signal generator module of the programmable electronic device and,
v) To generate the neurostimulation signal to the plurality of stimulation electrodes activating the afferent pathways.

19. Method for monitoring and suppression of pathological tremor via the stimulation of the afferent pathways to the brain of the user, making use of the device described in claim 3, characterized by comprising the following stages:
i) characterizing the tremor by means of a plurality of bioelectric sensors generating a bioelectrical characterization of the tremor, biomechanical sensors generating a biomechanical characterization signal of the tremor, or a combination thereof, wherein the bioelectrical, biomechanical or both characterization signals comprises at least the instantaneous frequency, phase or amplitude of the tremor sending these bioelectrical, biomechanical or both characterization signals to the control, acquisition and processing module of the programmable electronic device;
ii) differentiating the tremor from the voluntary movement of the user through a conventional analysis of the bioelectrical, biomechanical or both characterization signals by the control, acquisition and processing module;
iii) calculating a neurostimulation signal from the signal characterization of tremor in the control, acquisition and processing module of the programmable electronic device;
iv) generating the neurostimulation signal through the signal generator module of the programmable electronic device and,
v) To generate the neurostimulation signal to the plurality of stimulation electrodes activating the afferent pathways.

20. Method for monitoring and suppression of pathological tremor via the stimulation of the afferent pathways to the brain of the user, making use of the device described in claim 4, characterized by comprising the following stages:
i) characterizing the tremor by means of a plurality of bioelectric sensors generating a bioelectrical characterization of the tremor, biomechanical sensors generating a biomechanical characterization signal of the tremor, or a combination thereof, wherein the bioelectrical, biomechanical or both characterization signals comprises at least the instantaneous frequency, phase or amplitude of the tremor sending these bioelectrical, biomechanical or both characterization signals to the control, acquisition and processing module of the programmable electronic device;
ii) differentiating the tremor from the voluntary movement of the user through a conventional analysis of the bioelectrical, biomechanical or both characterization signals by the control, acquisition and processing module;
iii) calculating a neurostimulation signal from the signal characterization of tremor in the control, acquisition and processing module of the programmable electronic device;
iv) generating the neurostimulation signal through the signal generator module of the programmable electronic device and,
v) To generate the neurostimulation signal to the plurality of stimulation electrodes activating the afferent pathways.
